(19) United States
(12) Patent Application Publication
(10) Pub. No.: US 2008/0193497 A1
(43) Pub. Date: Aug. 14, 2008

(54) Hydrophilic Coating of a Water-Swellable Hydrophilic Matrix and an Anti-Microbial Polymer

(76) Inventors: Peter Samuelsen, Rungsted Kyst (DK); Soeren Kristiansen, Hundested (DK)

Correspondence Address: JACOBSON HOLMAN PLLC, 400 SEVENTH STREET N.W., SUITE 600, WASHINGTON, DC, 20004, US

(21) Appl. No.: 11/587,998

(22) PCT Filed: Apr. 29, 2005
(86) PCT No.: PCT/DK05/00293

§ 371 Date: Feb. 7, 2008

(30) Foreign Application Priority Data
Apr. 30, 2004 (DK) PA 2004 00678

(51) Int. Cl. A01N 37/20; A01N 25/10; A61L 29/08; A01P 1/00; A61L 29/16

(52) U.S. Cl. 424/405; 424/78.37; 424/78.08

(57) ABSTRACT

The present invention relates to a hydrophilic coating of a water-swellable hydrophilic matrix having incorporated therein an anti-microbial polymer, said anti-microbial polymer carrying pendant groups providing the anti-microbial effect, said pendant groups being selected from primary amino groups, secondary amino groups, tertiary amino groups, quaternary ammonium groups, imino groups, and phosphonium groups.

### FIELD OF THE INVENTION

The present invention relates to a hydrophilic coating of a water-swellable hydrophilic matrix having incorporated therein an anti-microbial polymer. The present invention also relates to objects coated with such hydrophilic coatings, in particular medical devices.

### BACKGROUND OF THE INVENTION

WO 02/17725 (D1) and WO 02/28927 (D2) disclose anti-microbial oligomers as well as polymer formulations comprising such oligomers combined with water-insoluble polymers. The oligomers and polymer formulations are used as coatings, varnishes and paints on a wide range of products, e.g. products within the field of medicine, e.g. coatings on contact lenses, catheters, etc.

WO 00/69264 (D3), WO 00/69925 (D4), WO 00/69933 (D5), and WO 00/69935 (D6) disclose anti-microbial polymers for producing anti-microbial polymer surfaces. The polymers are used as coatings on, or are grafted onto, a wide range of products, e.g. products within the field of medicine, e.g. coatings on contact lenses, catheters, etc.

### BRIEF DESCRIPTION OF THE INVENTION

D1-D6 individually represent close prior art. The underlying problem to be solved in view of the prior art is to provide an anti-microbial effect to an object, where the effect is uniform over a predetermined period of time and not solely dependent on direct contact between the microbe and the surface of the object.

None of the references D1-D6 anticipate the incorporation of an anti-microbial polymer (or oligomer) into a water-swellable hydrophilic matrix. On the contrary, D1-D2 both suggest that the water-soluble oligomer may be used alone, i.e. the oligomer may be washed away from the surface of the object fairly rapidly, or that the oligomer may be combined with a water-insoluble polymer. In the latter case, the oligomer needs to be leached out of a water-insoluble polymer. Leaching out will be become slower over time due to the formation of a leaching layer, i.e. the anti-microbial effect will be non-uniform. D3-D6 all suggest that the water-insoluble polymers or such polymer chains grafted to a surface are used to provide an anti-microbial surface. Although such surfaces are more or less permanently anti-microbial, the effect requires direct contact between the microbes and the surface.

Thus, the present invention provides an elegant solution to the above problem, cf. claim 1.

The invention further provides an object, cf. claim 14.

### DETAILED DESCRIPTION OF THE INVENTION

The Hydrophilic Coating

As mentioned above, the present invention relates to a hydrophilic coating of a water-swellable hydrophilic matrix having incorporated therein an anti-microbial polymer, said anti-microbial polymer carrying pendant groups providing the anti-microbial effect, said pendant groups being selected from primary amino groups, secondary amino groups, tertiary amino groups, quaternary ammonium groups, imino groups, and phosphonium groups.

Anti-Microbial Polymer

An important feature of the invention is the anti-microbial polymer which is incorporated in the hydrophilic matrix. The anti-microbial polymer is one which is carrying pendant groups providing the anti-microbial effect, said pendant groups being selected from primary amino groups, secondary amino groups, tertiary amino groups, quaternary ammonium groups, imino groups, and phosphonium groups. It should—of course—be understood that two or more such polymer may be used in combination.

As used herein with respect to the presence of the anti-microbial polymer in the hydrophilic coating, the term “incorporated” means that the anti-microbial polymer may be covalently bonded to the hydrophilic matrix or it may not be covalently bonded to the hydrophilic matrix.

In a preferred embodiment, all or an essential part of the anti-microbial polymer present in the hydrophilic coating is not covalently bonded to the hydrophilic matrix.

In another embodiment, all or an essential part of the anti-microbial polymer present in the hydrophilic coating is covalently bonded to the hydrophilic matrix.

The anti-microbial polymer typically has a weight average molecular weight in the range of 2,000-500,000, such as 4,000-250,000 or 4,000-50,000 or 50,000-250,000.

Examples of polymers carrying primary amino groups (—NH₂) are those incorporating monomers selected from the group consisting of 1-amino-propen, methacrylic acid-2-aminoethylester-hydrochloride, acrylic acid-3-aminopropyl ester, aminopropylmethacryl-amide, aminoethylvinyl ether, and 3-aminopropylvinyl ether.

Secondary amino groups may be of the type (—NHR¹) where R¹ is selected from the group consisting of C₁₋₆-alkyl, aryl, aryl-C₁₋₆-alkyl (such as benzyl), heteroaryl, and heteroaryl-C₁₋₆-alkyl (such as heteroaryl-methyl). Examples of polymers carrying secondary amino groups (—NHR¹) are those incorporating monomers selected from the group consisting of 1-(N-methyl)amino-propen, methacrylic acid-2-(N-methyl)aminoethylester-hydrochloride, acrylic acid-3-(N-methyl)aminopropyl ester, (N-methyl)aminopropylmethacrylamide, (N-methyl)aminoethylvinyl ether, and 3-(N-methyl)aminopropylvinyl ether.

Tertiary amino groups may be of the type (—N(R¹)₂) wherein each R¹ independently is selected as defined above. Examples of polymers carrying tertiary amino groups (—N(R¹)₂) are those incorporating monomers selected from the group consisting of 1-(N,N-dimethyl)amino-propen, methacrylic acid-2-(N,N-dimethyl)aminoethylester-hydrochloride, acrylic acid-3-(N,N-dimethyl)aminopropyl ester, (N,N-dimethyl)aminopropylmethacrylamide, (N,N-dimethyl)aminoethylvinyl ether, and 3-(N,N-dimethyl)aminopropylvinyl ether.

Quaternary ammonium groups may be of the type (—N⁺(R¹)₃) wherein each R¹ independently is selected as defined above. Examples of polymers carrying quaternary ammonium groups (—N⁺(R¹)₃) are those incorporating monomers corresponding to those defined above for tertiary amines. For this series, the counter-ion (the anion) to the ammonium group may any conventionally used counter-ion, or may be selected from anions which in themselves provides an anti-microbial effect.

Imino groups may be of the type (—N═C(R²)₂) wherein each R² independently is selected from hydrogen and the groups defined above for R¹. Examples of polymers carrying imino groups (—N═C(R²)₂) are those incorporating monomers corresponding to those defined above for primary amines condensed with aldehydes or ketones.

Phosphonium groups may be of the type (—P⁺(R³)₃) wherein R³ is independently selected from the groups defined for R¹ above. Examples of polymers carrying phosphonium groups (—P⁺(R³)₃) are those incorporating monomers selected from the group consisting of trimethylphosphoniumethylacrylate and trimethylphosphoniumethylmethacrylate. In this series, the counter-ion to the phosphonium groups may be selected as defined above for the quarternary ammonium group.

In a preferred embodiment, the anti-microbial polymer includes units derived from monomers selected from the group consisting of methacrylic acid-2-tert-butylaminoethylester, methacrylic acid-2-diethylaminoethylester, methacrylic acid-2-dimethylaminoethylester, methacrylic acid-2-diethylaminomethylester, acrylic acid-2-tert.-butylaminoethylester, acrylic acid-3-dimethylaminopropylester, acrylic acid-2-diethylaminoethylester, acrylic acid-2-dimethylaminoethylester, dimethylaminopropylmethacrylamide, diethylamino-propylmeth-acrylamide, acrylic acid-3-dimethylaminopropylamide, 2-methacryloyloxyethyltrimethyl-ammonium methosulphate, methacrylic acid-2-diethylaminoethylester, 2-methacryloyloxy-ethyltrimethylammonium chloride, 3-methacryloylaminopropyltrimethylammonium chloride, 2-methacryloyloxyethyltrimethylammonium chloride, 2-acryloyloxyethyl-4-benzoylbenzyldimethylammonium bromide, 2-methacryloyloxyethyl-4-benzoylbenzyldimethylammonium bromide, allyltriphenylphosphonium bromide, allyltri-phenylphosphonium chloride, 2-acrylamido-2-methyl-1-propanesulfonic acid, 2-diethylamino-ethylvinylether and/or 3-aminopropylvinylether.

Other monomers may be used as the balance in such anti-microbial polymers. Examples hereof are acrylic acid, methyl acrylate, methacrylic acid, methyl methacrylic acid, etc.The anti-microbial polymer may thus be selected from the group consisting of polymethacrylates and polyacrylates, carrying the above mentioned pendant groups.

More particular examples of anti-microbial polymers are those produced by polymerisation of one of several monomers as disclosed in patents DE 199 21 894 A1, DE 199 21 897 A1, DE 199 21 898 A1, DE 199 21 900 A1 and WO 02/17725 A1 or equivalent, and commercial products such as AMINA® T 100 (Degussa).

The anti-microbial polymer may be introduced to the hydrophilic matrices by different approaches but preferably through direct formulation into the final mixture of the matrix, as will be described in detail further below.

Water-Swellable Hydrophilic Matrix

The most fundamental constituent of the hydrophilic coating is the water-swellable hydrophilic matrix which has incorporated therein the anti-microbial polymer.

In the present context, the term “water-swellable” means that the dry polymer is able to absorb more than 30% of its weight in pure water. However, preferably the dry polymer shall be able to absorb at least 100% by weight in water.

In the present context a disintegrating swellable hydrophilic matrix means a matrix that will be able to pick up water to the characteristics of being water swellable before it starts to disintegrate i.e. its boundaries loose integrity.

Thus, in one embodiment, the weight ratio between the non-swollen and the instantly swollen (typically within 90 seconds) coating will be at least 1:1.3, e.g. at least 1:2.

In some further interesting embodiments, the ratio of the thickness of the hydrophilic coating in water swollen form (H_{water}) to the thickness of the hydrophilic coating in dry form (h_{dry}) is at least 2:1, such as at least 3:1.

The matrix typically comprises at least one water-swellable hydrophilic polymer which provides the water-swellability. In most instances, the matrix comprises at least 50% by solids weight of hydrophilic water-swellable polymers, such as at least 80% or 90% or more by solids weight of water-swellable hydrophilic polymers.

The term “hydrophilic” means in this context any organic or polymeric compound which is soluble in water in concentrations higher than 100 g/L.

Examples of water-swellable hydrophilic polymers are those selected from polyvinyl pyrrolidone, polyvinyl alcohol, poly(meth)acrylic acid, poly(meth)acrylic amides, polyethylene glycol, carboxymethylcellulose, cellulose acetate, cellulose acetate propionate, chitosan, polysaccharides; and homopolymers or copolymers of two or more of the monomers: N-vinylpyrrolidone, vinyl alcohol, (meth)acrylic acid, (meth)acrylic amides, (meth)acrylic esters (such as hydroxyethyl methacrylate), maleic anhydride, maleimide, methyl vinyl ether, alkyl vinyl ethers, and other unsaturated compounds. Furthermore, the hydrophilic water-swellable polymer may be any derivative or any blend of these homopolymers or copolymers.

Most preferably, the hydrophilic polymer of the coating is selected from the group consisting of polyethylene glycol, polyvinylpyrrolidone and copolymers including polyvinylpyrrolidone, e.g. polyvinylpyrrolidone-vinyl acetate copolymers. When using the pure polyvinylpyrrolidone (poly(N-vinyl-2-pyrrolidone); PVP), various chain lengths may be selected each giving various characteristics to the coating. Typically, such polyvinylpyrrolidone polymers have a number average molecular weight of above 100,000. As an example, PVP K-90 with a molecular weight of 1,200,000 can be selected but other types of PVP with other molecular weights may also be used.

In one interesting embodiment, the matrix comprises at least 50% by solids weight of one or more hydrophilic polymers selected from the group consisting of polyethylene glycol, polyvinylpyrrolidone and copolymers including polyvinylpyrrolidone.

It is generally preferred that the matrix consists of non-cross-linked polymers.

In particular, the matrix should be at least partly soluble in water.

One way of determining the water-solubility is to store an amount of the matrix at 20° C. in 1 L of pure water under stirring which over 24 hours at 20 degree C. still leaves a non-dissolved residue. The solubility of the matrix is the amount dissolved in g/L per gram matrix material and determine the time at which the matrix has fully disintegrated. Preferably, the matrix will fully disintegrate upon storage for 24 hours at 20° C. in 1 L of pure water per gram matrix.

The coating should of course not disintegrate immediately, thus the type of polymer, the chain length, etc. should preferably be selected so that the hydrophilic matrix disintegrates sufficiently slowly, i.e. such that it has not fully disintegrated upon storage for 2 hours at 20° C. in 1 L of pure water per gram matrix.

The Coating of the Matrix and Anti-Microbial Polymer

In order to obtain an even release profile for the anti-microbial polymer and the matrix, the matrix and the anti-microbial polymer preferably form a homogeneous water-swellable coating.

The present invention is particularly relevant for fairly thick coatings as those useful for medical devices, such as catheters, e.g. urinary catheters. Thus, in an interesting embodiment, the thickness in dry form (h_{dry}) of the hydrophilic coating is at least 3 μm, such as 6 μm, e.g. 3-1500 μm, such as 6-500 μm.

The Object

Substrate

The hydrophilic coating is most often applied to a substrate so as to provide anti-microbial properties to an object. Such objects, e.g. medical devices and medical device elements, can be formed from a variety of types of basic materials, such as plastics, metals, glass, ceramics, etc. Typical examples of plastic materials for medical devices are polymers such as polyurethanes and copolymers thereof, or polyether block amides such as Pebax™ or other polymer materials including polyvinyl chloride, polyamide, silicone, styrene-ethylene/butylene-styrene block copolymers (SEBS), styrene-isoprene-styrene block copolymers (SIS), styrene-ethylene/propylene-styrene block copolymers (SEPS), ethylene-vinyl acetate copolymers (EVA), polyethylene (PE), metallocene-catalyzed polyethylene, and copolymers of ethylene and propylene or mixtures of such. For some of the combinations of substrate polymers and hydrophilic coatings (e.g. polyethylene as a substrate polymer and PVP as the hydrophilic polymer) a primer coat (e.g. a PVC or polyurethane solution) may advantageously be applied before application of the polymer solution. Still further the substrate may be moulds of metal, glass or equivalent. The substrate will inherently normally be hydrophobic. Currently very relevant materials for medical devices are polyurethanes and copolymers thereof.

Within the present context, the medical device has on at least a part of the surface thereof (i.e. on at least a part of the surface of the basic material) a hydrophilic coating as defined herein. In some embodiments, the hydrophilic coating is applied to the full (outer) surface of the substrate polymer, and in some other embodiments, only to a part of the surface. In the most relevant embodiments, the coating is applied to at least a part of the surface (preferably the whole surface) of a medical device that—upon proper use—comes into direct contact with body parts of the person for which the medical device is intended.

The substrate is preferably at least part of an object, e.g. a medical device. Thus, a further aspect of the present invention relates to an object comprising a substrate material and a coating covering at least a part of the surface of said substrate material, said coating being a hydrophilic coating as defined herein. As mentioned above, the substrate material is typically selected from the group consisting of plastics, metals, glass, and ceramics.

Preferably, the object is a medical device or a medical device element, preferably a medical device or medical device element selected from the group consisting of catheters, endoscopes, laryngoscopes, tubes for feeding, tubes for drainage, guide wires, condoms, urisheaths, barrier coatings, stents and other implants, extra corporeal blood conduits, membranes, blood filters, devices for circulatory assistance, dressings for wound care, and ostomy bags. Currently most relevant medical devices or medical device elements are catheters and catheter elements.

Preparation of the Hydrophilic Coating

A hydrophilic coating defined herein may be prepared by one of the following general procedures.

Equilibration of Existing Hydrophilic Matrix

One way of providing the hydrophilic coating of the invention is to dissolve the anti-microbial polymer in a water phase, and equilibrate an already existing hydrophilic matrix with this solution so as to allow the anti-microbial polymer to penetrate the hydrophilic matrix.

Thus, in one variant, the aqueous solution used to equilibrate the matrix may comprise:

10⁻⁵-20 wt/vol % of the anti-microbial polymer,

0-10 wt/vol % of polyvinylpyrrolidone and/or of polyethylenglycol,

0-50 wt/vol % of one or more osmolality increasing agents, and

20-99.5 wt/vol % of solvent.

The aqueous solution should preferably have a pH in the range of 3.5-8.5.

Thus, an object with a hydrophilic coating according to the invention may be provided through a method comprising the steps of:

(I) providing a substrate polymer having the substrate surface,

(II) providing a polymer solution comprising one or more hydrophilic polymers,

(III) applying said polymer vehicle solution to said substrate,

(IV) optionally evaporating at least a part of the vehicle from said polymer solution present on said substrate polymer surface, and

(VI) adding a solution containing the anti-microbial polymer to the hydrophilic matrix.

An advantage of this method is that the step of allowing the anti-microbial to penetrate the hydrophilic matrix does not necessitate any changes in the procedure for establishing the hydrophilic matrix as such.

Preparation of Matrix with Anti-Microbial Polymer

In another variant, the anti-microbial polymer may be added to the polymer solution intended to produce the matrix on the medical device. In particular, the ingredients required for the formation of the matrix may be solubilized in water or any other aqueous mixture or organic solvent. The polymer solution may be based on water, or, preferably, ethanol or other alcohols, or mixture thereof. Currently preferred solvents include those selected from N-methyl-2-pyrrolidone, dimethyl sulfoxide, acetone, 1,3-dioxolane and dimethyl formamide. However, any solvent can in principle be used for the vehicle. Other options may therefore include methylethylketon, diethylether, dioxan, hexan, heptan, benzol, toluol, chloroform, dichlormethan, tetrahydrofuran and acetonitril, 1,3-dioxolane and other ethers, acetone and other ketones, dimethylsulfoxide and other sulfoxides, dimethyl formamide and other amides, N-methyl-2-pyrrolidone and other lactams, glycols, glycol ethers, glycol esters, other esters, amines, heterocyclic compounds, alkylated urea derivatives, liquid nitriles, nitroalkanes, haloalkanes, haloarenes, trialkyl phosphates, dialkyl alkanephosphonates, and other commonly known organic solvents. The preferred solvents may either be used singly or in combination. Typically, the solvent(s) constitute(s) 50-95% by weight of the polymer solution. In practical, the choice of mixture may be selected on the basis of the substrate since specific organic solvents may be required for the partly solubilization of the substrate surface. For example in a preferred embodiment of the invention, the vehicle may comprise mixtures of water, ethanol or ethanol and N-methyl-2-pyrrolidone.

Thus, in one embodiment of the invention the polymer solution used to produce the matrix may comprise:

10⁻⁵-20 wt/vol % of the anti-microbial polymer,

0.2-50 wt/vol % of a hydrophilic polymer,

0-40 vol/vol % of one or more plastizers,

0-50 wt/vol % of one or more osmolality increasing agents, and

20-99.5 wt/vol % of solvent.

The polymer solution should preferably have a pH in the range of 3.5-8.5.

The preferred plasticizers are glycols i.e. glycerol, polyethylene glycols, polypropylene glycols or acetyl triethyl citrate, dimethyl sulfone, ethylene carbonate, glycerol diacetate, glycerol triacetate, hexamethylphosphoramide, isophorone, methyl salicylate, N-acetyl morpholine, propylene carbonate, quinoline, sulfolane, triethyl citrate, and triethyl phosphate. Particular examples are acetyl triethyl citrate. The plasticizers may be used singly or in combination. The plasticizer(s) preferably constitute(s) 1-40% by weight of the polymer solution.

The volatile vehicle solution comprising the above ingredients can be applied to the substrate surface by conventionally techniques (dipping, spraying, incubation, rolling etc.) and may subsequently be dried by evaporation of solvents. The matrix can then later be swollen with water or an aqueous solution prior to use in order to give low friction properties.

Thus, an object with a hydrophilic coating according to the invention may be provided through a method comprising the steps of:

(I) providing a substrate polymer having the substrate surface,

(II) providing a polymer solution comprising one or more hydrophilic polymers and an anti-microbial polymer,

(III) applying said polymer vehicle solution to said substrate, and

(IV) optionally evaporating at least a part of the vehicle from said polymer solution present on said substrate polymer surface.

Step (I)

As mentioned above, the substrate may be the native surface of a medical device or another object, or may be surface treated so as to facilitate strong bonding of the hydrophilic matrix to the substrate. The surface of the substrate may be the complete physical surface or a fraction thereof. For instance with many medical devices, it is only necessary to coat the part of the substrate polymer surface, which comes into direct contact with the surface of living tissue when in use. Thus, the step of providing a substrate having the substrate surface will be evident for the person skilled in the art. In a special embodiment, the surface of the substrate can be in the form of a mould. After formation of the matrix, the matrix is removed from the mould and the mould may therefore not be an integrated part of the final medical device.

Step (II)

The choice of hydrophilic polymer and optionally monomers, vehicle including water, solvent(s) and plasticizer(s) and additives including the anti-microbial oligomer or polymer is described above. The solution may be prepared by mixing the components of the vehicle with the hydrophilic polymer in order to obtain the polymer solution. The mixing order is not particularly critical as long as a homogeneous (and possibly clear) solution is obtained. Thus, the step of actual preparation of the polymer solution may be evident for the person skilled in the art in view of the above directions with respect to choice of vehicle components.

Step (III)

Application of the polymer solution to said substrate surface is conducted following conventional methods such as bar coating, reverse roll coating, dip coating, spray coating, application by means of brushes, rollers, etc., as will be evident for the person skilled in the art. With due consideration of the production process, it is preferred that the application of the polymer to the substrate polymer surface is performed by dipping the medical device (or the relevant surface thereof) into the polymer solution when non-planar and using bar coating with or for planar species.

The polymer solution is applied to a substrate polymer surface in one or more steps, suitably in one single application step, such as in a one-dip process. It should however be understood that the substrate polymer may be primed in one or more preceding step and that such a preceding step may be applied in addition to a single application step (one-dip process).

Step (IV)

After application of the polymer solution to the substrate surface, any organic solvent is evaporated from the polymer solution present on said substrate polymer surface. The solvents may be removed by passive evaporation, by leading a stream of air over the surface of the substrate, or by applying a reduced pressure over the surface of the substrate.

Furthermore, it may be necessary or desirable to increase the temperature of the substrate or the air surrounding the substrate to speed up the evaporation process. Preferably, the evaporation process is facilitated by drying the substrate with the polymer solution at a temperature of between 25-100° C. depending on the thermostability of the substrate and the polymer. Typically, the substrate (e.g. a medical device) is dried in an oven. When water constitutes the entire solvent system or a part of the solvent system, preferably no or only part of this is evaporated as a rehydration step is eliminated for instance in case of sheet hydrogels.

Combination

Alternatively, in a third embodiment both approaches may be combined; the anti-microbial polymer may present in the solution required to equilibrate the matrix and present in the polymer solution required for the production of the hydrophilic matrix.

Thus, an object with a hydrophilic coating according to the invention may be provided through a method comprising the steps of:

(I) providing a substrate polymer having the substrate surface,

(II) providing a polymer solution comprising one or more hydrophilic polymers and an antimicrobial polymer

(III) applying said polymer vehicle solution to said substrate,

(IV) optionally evaporating at least a part of the vehicle from said polymer solution present on said substrate polymer surface, and

(VI) adding a solution containing the anti-microbial polymer to the hydrophilic matrix.

After-Treatment

The hydrophilic coating may afterwards be dried or may be sizing and packed in a suitable form. For medical devices, the package is preferably sterilized subsequent to packing.

The hydrophilic matrix in form of a coating become highly lubricious when wet as the coating takes up a significant amount of water, which leaves a non-bonded layer of free water molecules at the surface of the coating. The non-bonding character of the surface water is believed to cause the low friction of the wet coating. Hence, the coating when applied to a biomedical or other device will improve biocompatibility and patient compliance. However, for most applications there will be high demands to the internal and the bonding strength for the coating. Thus, in a preferred embodiment of the invention the coating on the medical device may be packed in an un-swollen—or dry—condition, and saturated with a solution preferentially water before use. The solution used to saturate the coating may be a part of the packing device and therefore provided by the manufacturer, or the solution may be added and provided by the end-user. Alternatively, the coating on the medical device may also be packed in a swollen state and the solution is therefore added by the manufacturer prior to the sterilization process.

### EXAMPLES

### Example 1

A hydrophilic coating on a catheter from PVC is produced from applying a first coating layer by immersion of the catheter into a mixture of 5.4 g of low viscosity nitrocellulose, 2.0 g dibutyl phthalate and 1.9 g polyvinylbutyral (PVB) in a mixed solvent comprising isopropanol, ethyl acetate, ethanol and acetone (6:25:18:1.5 vol/vol) adding up to 100 g. After drying in 5 minutes at 65° C. and additional outer layer was applied in a second coating step by dipping in a solution of 6.6 g of polyvinylpyrrolidone (Plasdone K 90) and 0.5 g antimicrobial polymer (Amina T 100) in a solvent of ethanol and N-vinyl pyrrolidone (80:20 vol/vol) adding up to 100 g. The catheter was allowed to dry 24 hours at 65° C.

### Example 2

A hydrophilic coating on a catheter form PVC is produced as of Example 1 with the addition of 1.5 g urea to the solution of the second coating step.

### Example 3

A hydrophilic coating on a catheter form PVC is produced as of Example 1 with the addition of 2.5 g sodium chloride finely milled to the solution of the second coating step. The second coating step is performed under gentle stirring of coating media.

### Example 4

6.0 g polyurethane (Desmodur L 2291, Bayer AG) composed by trimerized diisocyanate of biuret is dissolved in 94.0 g methylene chloride. A urinary polyurethane catheter is dipped in this solution for 30 seconds, whereafter the the catheter is dried at 65° C. for one minute. In a second coating step the catheter is dipped in a solution of 33 g polyvinylpyrrolidone (Plasdone K 90 (ISP)/MW 90,000), 1.0 g Amina T 100 and 66 g methylene chloride. The catheter is allowed to dry at ambient temperature followed by a one hour drying at 80° C.

### Example 5

A coating as of Example 4 to which in a third step an extra coating layer is applied. The third step consists of a coating solution containing 20% by weight of sodium chloride, 5% of PVP (Plasdone K25/MW 25.000) and 0.2 g Amina T 100 in same solvent. A 24 hours dying step concludes the processing of the catheter.

### Example 6

A PVC tubing is dip coated in a polymer solution of 5.4 g low viscosity nitrocellulose, 2.0 g dibutylphthalate, 1.5 g camphor in a solvent mixture of 36 ml toluene, 13.1 ml butylacetate, 5.9 ml isopropanol, 25.4 ml ethyl acetate, 18.1 ml ethylalcohol, and 1.5 ml acetone. The tube is dried for 5 minutes at 65° C. It is thereafter dipped in a hydrophilic polymer solution containing 6.6 g polyvinylpyrrolidone (Plasdone K 90), 0.6 g Amina T 100, 64 ml ethylalcohol, 24 ml ethyl acetate, 13 ml dimethylformamide and finally dried at 80° C. for 24 hours.

1. A hydrophilic coating of a water-swellable hydrophilic matrix having incorporated therein an anti-microbial polymer, said anti-microbial polymer carrying pendant groups providing the anti-microbial effect, said pendant groups being selected from primary amino groups, secondary amino groups, tertiary amino groups, quaternary ammonium groups, imino groups, and phosphonium groups.

2. The hydrophilic coating according to claim 1, wherein the anti-microbial polymer has a weight average molecular weight in the range of 2,000-500,000.

3. The hydrophilic coating according to claim 1, wherein the anti-microbial polymer includes units derived from monomers selected from the group consisting of methacrylic acid-2-tert-butylaminoethylester, methacrylic acid-2-diethylaminoethylester, methacrylic acid-2-dimethylaminoethylester, methacrylic acid-2-diethylaminomethylester, acrylic acid-2-tert.-butylaminoethylester, acrylic acid-3-dimethylaminopropylester, acrylic acid-2-diethylaminoethylester, acrylic acid-2-dimethylaminoethylester, dimethylaminopropylmethacrylamide, diethylamino-propylmethacrylamide, acrylic acid-3-dimethylaminopropylamide, 2-methacryloyloxyethyltrimethylammonium methosulphate, methacrylic acid-2-diethylaminoethylester, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-acryloyloxyethyl-4-benzoylbenzyldimethylammonium bromide, 2-methacryloyloxyethyl-4-benzoyldimethylammonium bromide, 2-methacryloyloxyethyl-4-benzoylbenzyldimethylammonium bromide, allyltriphenylphosphonium bromide, allyltriphenylphosphonium chloride, 2-acrylamido-2-methyl-1-propanesulfonic acid, 2-diethylaminoethylvinylether and 3-aminopropylvinylether.

4. The hydrophilic coating according to claim 1, wherein the anti-microbial polymer is selected from the group consisting of polymethacrylates and polyacrylates.

5. The hydrophilic coating according to claim 1, wherein the weight ratio between the non-swollen and the instantly swollen coating will be at least 1:1.3.

6. The hydrophilic coating according to claim 1, wherein the water-swellable hydrophilic matrix comprises at least one hydrophilic polymer selected from the group consisting of polyvinyl pyrrolidone, polyvinyl alcohol, polyacrylic acid, polymethacrylic acid, polyacrylic amides, polymethacrylamide, polyethylene glycol, carboxymethylcellulose, cellulose acetate, cellulose acetate propionate, chitosan, any other polysaccharides, and grafts or copolymers of any of these polymers such as copolymers with maleic anhydride, succinic anhydride or the corresponding acids.

7. The hydrophilic coating according to claim 6, wherein the matrix comprises at least 50% by polymer solids weight of one or more hydrophilic polymers selected from the group consisting of polyethylene glycol, polyvinylpyrrolidone and copolymers including polyvinylpyrrolidone.

8. The hydrophilic coating according to claim 1, wherein the matrix consists of non-cross-linked polymers.

9. The hydrophilic coating according to claim 1, wherein the matrix is at least partly soluble in water.

10. The hydrophilic coating according to claim 9, wherein the matrix will fully disintegrate upon storage for 24 hours at 20° C. in 1 L of pure water per gram matrix.

11. The hydrophilic coating according to claim 1, wherein the matrix has not fully disintegrated upon storage for 2 hours at 20° C. in 1 L of pure water per gram matrix.

12. The hydrophilic coating according to claim 1, wherein the matrix and the anti-microbial polymer form a homogeneous water-swellable coating.

13. The hydrophilic coating according to claim 1, wherein the thickness in dry form (h_{dry}) of the hydrophilic coating is at least 3 μm.

14. An object comprising a substrate material and a coating covering at least a part of the surface of said substrate material, said coating being a hydrophilic coating as defined in claim 1.

15. The object according to claim 14, wherein the substrate material is selected from the group consisting of plastics, metals, glass, and ceramics.

16. The object according to claim 14, which is a medical device or a medical device element.

17. The object according to claim 16, wherein the medical device or medical device element is selected from the group consisting of catheters, endoscopes, laryngoscopes, tubes for feeding, tubes for drainage, guide wires, condoms, urisheaths, barrier coatings, stents and other implants, extra corporeal blood conduits, membranes, blood filters, devices for circulatory assistance, dressings for wound care, and ostomy bags.

18. The object according to claim 17, wherein medical device or medical device element is a catheter or catheter element.
